(19) United States
(12) Patent Application Publication
(10) Pub. No.: US 2012/0006330 A1
(43) Pub. Date: Jan. 12, 2012

(54) Ventilator Coupling Lock for Tracheostomy Tube

(76) Inventor: Ben John Barbot, Richmond, VA (US)

(21) Appl. No.: 12/831,150

(22) Filed: Jul. 6, 2010

(51) Int. Cl. A61M 16/04

(52) U.S. Cl. 128/207.14

(57) ABSTRACT

The embodiments pertain to tracheostomy tubes with an outer cannula and a disposable inner cannula which is connected to a ventilator coupling and the other end of the ventilator coupling connected to the ventilator tubing. The existing connection between the disposable inner cannula and the ventilator coupling is made by simple insertion of the disposable inner cannula into the ventilator coupling and unintentional disconnections do occur.

Embodiments consist of locking or securely fastening the connection between the disposable inner cannula and the ventilator coupling. One embodiment consists of level arms that are mounted on a ventilator coupling and a retaining collar mounted on a disposable inner cannula to securely fasten the ventilator coupling to the disposable inner cannula. This will prevent accidental or unintentional disconnection and thereby the patient's health will not be compromised nor will it deteriorate and death may be prevented. Also, the embodiments enable an easy manual release or disconnection of the disposable inner cannula and the ventilator coupling. The disposable inner cannula and the ventilator coupling are inexpensively constructed using polymer and injection molding process.

### BACKGROUND

Tracheostomy tubes with an outer cannula and a disposable inner cannula and also ventilator couplings have been used for some time to provide bypass supply of air to a patient unable to breathe on their own due various medical reason such as paralysis, physical trauma or an obstruction in the throat. U.S. Pat. No. 4,315,505 (1982), is the most commonly used tracheostomy tube. Ventilator couplings are employed to connect the disposable inner cannula to the tubing of a ventilator or respiratory device to assist the patient's breathing. Tracheostomy tubes and ventilator couplings are inexpensively constructed using polymer and injection molding process.

The disposable inner cannula can become blocked from an accumulation of mucus and/or phlegm and therefore the disposable inner cannula is easily releasable from the outer cannula and disconnected from the ventilator coupling to facilitate replacement, cleaning or clearing.

The disposable inner cannula is fastened or connected to the ventilator coupling by simply inserting the disposable inner cannula into the ventilator coupling. Friction between these two cylindrical surfaces is the only force resisting and preventing the disconnection of the disposable inner cannula from the ventilator coupling. Mucus is normally present in this environment and the presence of mucus between this connection can cause a lubricating effect and thereby reduce the friction between these two cylindrical plastic surfaces.

While in place within the patient's trachea, the disposable inner cannula can unintentionally disconnect from the ventilator coupling due to many reasons including patient coughing and movement. This disconnection causes the patient's assisted ventilation to cease until the attending nurse or health care worker reconnects the ventilator coupling to the disposable inner cannula. This disruption of the patient's assisted ventilation may cause the patient's health to be compromised, deteriorate and can cause death. This disconnection has been somewhat of a common occurrence for some time at health care facilities.

### SUMMARY

In accordance with one embodiment, a ventilator coupling lock for a tracheostomy tube comprises of a secure method or mechanism which secures and locks the disposable inner cannula of a tracheostomy tube to the ventilator coupling and which also has an easily releasable disconnect to disconnect the ventilator coupling from the of the disposable inner cannula.

### ADVANTAGES

Accordingly, one advantages is to provide a removably secure ventilator coupling to disposable inner cannula connection which will prevent the accidental or unintentional disconnection of the ventilator coupling to disposable inner cannula connection. This preventive measure will eliminate the disruption to the patient's assisted ventilation, the patient's health will not be compromised and deteriorated and death may be prevented. The attending nurse or health care worker will not need to restore the connection from the ventilator coupling to disposable inner cannula connection. Also an easy releasable disconnection of the disposable inner cannula to the ventilator coupling will facilitate the removal, replacement and cleaning of the disposable inner cannula.

### DRAWINGS

### Figures

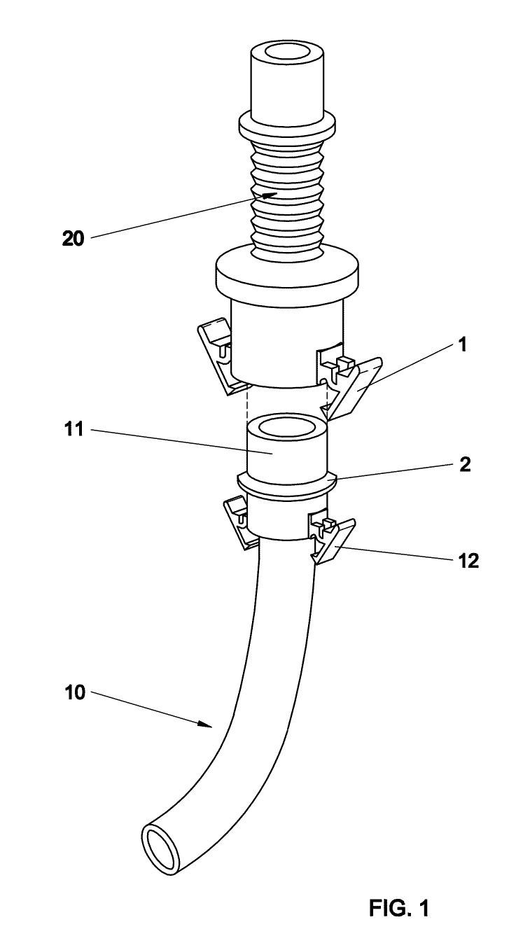

FIG. 1 is a perspective view of the ventilator coupling and disposable inner cannula removably secure connection. A tracheostomy tube consists of an outer cannula and a disposable inner cannula and the disposable inner cannula is adapted with a retaining collar and the ventilator coupling is adapted with lever arms. Together the retaining collar and lever arms provide a secure, locking and also a releasable disconnection method in accordance with this embodiment. The level arms securely lock down on the retaining collar and also can be easily released.

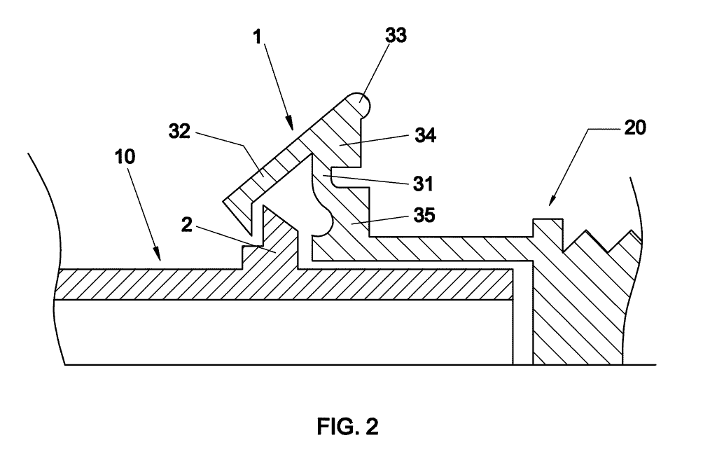

FIG. 2 is a partial sectional view taken through the ventilator coupling and the connector of the disposable inner cannula illustrating details of the lever arms and the retaining collar in accordance with one embodiment.

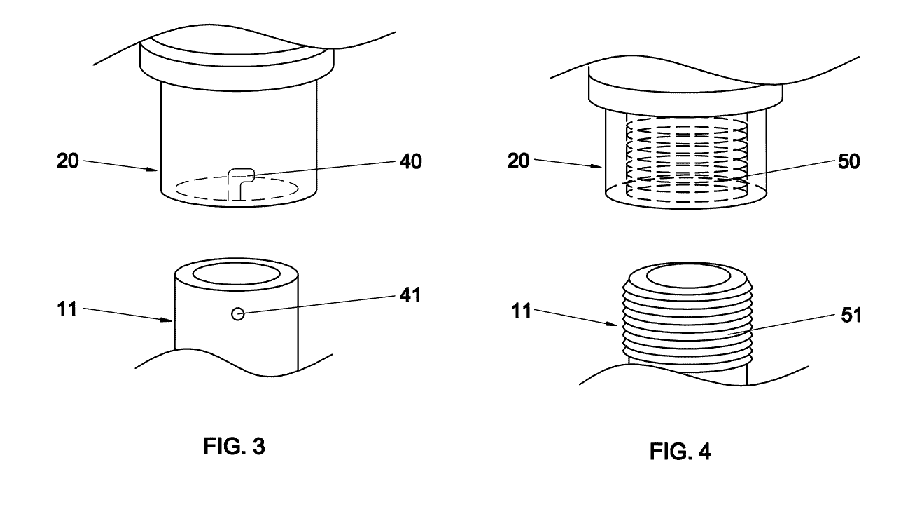

FIG. 3 is a perspective view of the ventilator coupling removably secure connection to disposable inner cannula using an internal groove and a retaining pin in accordance with one embodiment.

FIG. 4 is a perspective view of the ventilator coupling removably secure connection to disposable inner cannula using outside and inside threads and a retaining collar in accordance with one embodiment.

Drawings - Reference Numerals

| | | | |
|---|---|---|---|
| 1 | lever arms | 2 | retaining collar |
| 10 | disposable inner cannula | 11 | connector |
| 12 | inner cannula level arms | 20 | ventilator coupling |
| 31 | hinges | 32 | hinges |
| 33 | handle ends | 34 | ribs |
| 35 | stop blocks | 40 | inner groove |
| 41 | retaining pin | 50 | inner threads |
| 51 | outer threads | | |

### DETAILED DESCRIPTION

### FIGS. 1 and 2—First Embodiment

One embodiment of the ventilator coupling lock for disposable inner cannula of a tracheostomy tube with an outer cannula and a disposable inner cannula is illustrated in FIG. 1 and FIG. 2.

This embodiment consists of level arms 1 which are mounted or molded on the ventilator coupling 20 and a retaining collar 2 which is mounted or molded on the connector 11 of the disposable inner cannula 10 of a tracheostomy tube with an outer cannula and a disposable inner cannula and used to securely fasten the ventilator coupling 20 to the disposable inner cannula 10 to prevent accidental disconnection and also allow for an easy manual release to facilitate the cleaning or replacement of the disposable inner cannula 10.

Referring to FIG. 1, the ventilator coupling 20 and the disposable inner cannula 10 are similar to those common used for some time except with the addition of the Lever Arms 1 and the retaining collar 2 of this embodiment. A disposable inner cannula 10 is comprised of the connector 11, the retaining collar 2 and the inner cannula level arms 12 and connects to the ventilator coupling 20 and is secured by the level arms 1 when engaged to the retaining collar 2 and the other end connects into the outer cannula of the tracheostomy tube. This embodiment includes the addition of molding a retaining collar 2 to the disposable inner cannula 10. A ventilator coupling 20 connects to the connector 11 of the disposable inner cannula 10 and the other end connects to the ventilator tubing. This embodiment includes the addition of molding Lever Arms 1 to the ventilator coupling 20. The ventilator coupling 20 is secured in place by means of the lever arms 1 which engage an annular retaining collar 2 located on the connector 11 of the disposable inner cannula 10.

Referring to FIG. 2, integrally formed on opposite sides of the ventilator coupling 20 are a pair of lever arms 1 which are biased forwardly or toward one another by accurate, resilient hinges 31. In their relaxed state, these hinges 31 cause the lever arms 1 to assume the position shown in FIG. 2. The lever arms 1 have locking ends 32 and handles ends 33. Each locking ends 32 engages the retaining collar 2 located on the connector 11 of the disposable inner cannula 10.

### OPERATION

### FIGS. 1 and 2—First Embodiment

To connect the ventilator coupling 20 to connector 11 of the disposable inner cannula 10, the locking ends 32 are manually spread apart by exerting an inward force from the fingers on the handle ends 33 of the lever arms 1 until the locking ends 33 of the lever arms 1 are separated by a distance greater than the outside diameter of the retaining collar 2 and thereby they can be advanced beyond the retaining collar 2. The ventilator coupling 20 is then advance slightly and the handle ends 33 released, permitting the hinges 31 to bias the locking ends 32 together in locking engagement with the retaining collar 2. The ventilator coupling 20 is easily unlocked or released from the coupling connector 11 of the disposable inner cannula 10 by reversing these simple steps.

The lever arms 1 have hinges 31, ribs 34 and stop blocks 35 to prevent damage due to backwards hyperextension.

### DESCRIPTION

### FIGS. 3 and 4—Additional Embodiments

Additional embodiments are shown in FIGS. 3 and 4. In FIG. 3, this embodiment consists of an inner groove 40 which is molded on the inside of the ventilator coupling 20 and a retaining pin 41 which is molded on the connector 11.

In FIG. 4, this embodiment consists of inner threads 50 which are molded on the inside of the ventilator coupling 20 and outer threads 51 which molded on the outside of the connector 11 of the disposable inner cannula 10.

### OPERATION

### FIGS. 3 and 4—Additional Embodiments

The manner of using the embodiment shown in FIG. 3 for the ventilator coupling lock for disposable inner cannula is identical to the current method of use except for the addition of the operation of the inner groove 40 and the retaining pin 41 of this embodiment.

To connect the ventilator coupling 20 to connector 11 of the disposable inner cannula 10, the ventilator coupling 20 and connector 11 are align so that the retaining pin 41 is aligned with the inner groove 40 and then inserted together with the retaining pin following the inner groove 40 until the inner movement is blocked by the curvature of the inner groove 40 and then twisted into a locking position until the movement stops. The ventilator coupling 20 is easily unlocked or released from the coupling connector 11 of the disposable inner cannula 10 by reversing these simple steps.

The manner of using embodiment shown in FIG. 4 for the ventilator coupling lock for disposable inner cannula is identical to the current method of use except for the addition of the inner threads 50 and the outer threads 51 of this embodiment.

To connect the ventilator coupling 20 to connector 11 of the disposable inner cannula 10, the ventilator coupling 20 and connector 11 are align and then threaded together until the movement of the threads become tight and stops. The ventilator coupling 20 is easily unlocked or released from the coupling connector 11 of the disposable inner cannula 10 by reversing these simple steps.

### CONCLUSION, RAMIFICATIONS AND SCOPE

Accordingly, the reader will see that, the ventilator coupling lock for disposable inner cannula of the various embodiments can be used to easily and conveniently secure and lock the ventilator coupling to the disposable inner cannula. The disposable inner cannula can also be easily manually released or unlocked and removed.

The removably secure lockable connection will prevent accidental or unintentional disruption of the patient's assisted ventilation and thereby the patient's health will not be compromised nor deteriorate and death may be prevented. The improvement described in the various embodiments have secure mechanisms to securely connect the ventilator coupling to the disposable inner cannula for the prevention of unintentional disconnection including accidental disconnection and also to easily release the connection.

I claim:

1. A ventilator coupling lock comprising:
a. a tracheostomy tube with an outer cannula and a disposable inner cannula for insertion into a patient's trachea through an opening in the neck to assist breathing with the disposable inner cannula adapted to be removably secured;
b. a ventilator coupling adapted to be removably secured;
c. a holdfast means for removably securing said ventilator coupling to said disposable inner cannula.

2. The ventilator coupling lock of claim 1 wherein said ventilator coupling has lever arms and the disposable inner cannula has a retaining collar for removably securing the ventilator coupling to the disposable inner cannula.

3. The ventilator coupling lock of claim 1 wherein said ventilator coupling has an inner groove and the disposable inner cannula has a retaining pin for removably securing the ventilator coupling to the disposable inner cannula.

4. The ventilator coupling lock of claim 1 wherein said ventilator coupling has inner threads and the disposable inner cannula has outer threads for removably securing the ventilator coupling to the disposable inner cannula.
